(19) United States
(12) Patent Application Publication
(10) Pub. No.: US 2026/0047937 A1
(43) Pub. Date: Feb. 19, 2026

(54) KNEE PROSTHESIS

(71) Applicant: Joint Development, Inc., Salt Lake City, UT (US)

(72) Inventors: Dermott J. McHugh, Anchorage, AK (US); Eric M. Dacus, Salt Lake City, UT (US); Chris A. Weaber, Sandy, UT (US); Andrea M. Cragen, Austin, TX (US)

(21) Appl. No.: 19/299,069

(22) Filed: Aug. 13, 2025

Related U.S. Application Data
(60) Provisional application No. 63/682,996, filed on Aug. 14, 2024.

(51) Int. Cl. A61F 2/38; A61F 2/30; A61L 27/06; A61L 27/56

(52) U.S. Cl. CPC A61F 2/3859; A61L 27/06; A61L 27/56; A61F 2002/30011; A61F 2002/30084; A61L 2400/12

(57) ABSTRACT

An embodiment utilizes Ion Beam Enhanced Deposited (IBED) TiN in orthopedics to improve the wear and durability of Titanium articular surfaces and to reduce the leaching of metal ions associates with Cobalt Chrome implants.

This application claims priority to U.S. Provisional Patent Application No. 63/682,996 filed on Aug. 14, 2024 and entitled “Knee Prosthesis”, the content of which is hereby incorporated by reference.

### BACKGROUND

Unicompartmental knees have been in use for many years, however clinical challenges remain: fixation, revision, infection, and the like. Both symmetric and asymmetric implant designs have been created utilizing a variety of materials to attempt to solve the problems of partial knee replacement. These designs have used both cemented and cementless fixation.

### BRIEF DESCRIPTION OF THE DRAWINGS

Features and advantages of embodiments of the present invention will become apparent from the appended claims, the following detailed description of one or more example embodiments, and the corresponding figures. Where considered appropriate, reference labels have been repeated among the figures to indicate corresponding or analogous elements.

FIG. 1A includes a perspective view of an embodiment. FIG. 1B includes a side view of the embodiment of FIG. 1A. FIG. 1C includes another perspective view of the embodiment of FIG. 1A. FIG. 1D includes a cross-sectional view taken at “Plane 4” of FIG. 1C. FIG. 1E includes an simplified abstract cross-section view from the area called out in FIG. 1D.

FIG. 2 includes an abstract cross-§ of a ion embedded porous surface in an embodiment.

FIG. 3 includes an image of a porous surface or region.

FIG. 4A includes an image of a CoCr plug with smooth and porous surfaces. FIG. 4B includes a scanning electron microscopic (SEM) image of a Ti porous coating. FIG. 4C includes a SEM image of a CoCr porous coating.

FIGS. 5A-5H present various views of an embodiment.

FIG. 6: (Top) Bright field TEM images of an entire sample for each coating type showing coarser grain structure in the PVD coatings, and increasing grain size in the bulk of the coating. (Middle) Bright field images of the coating-substrate interface. The vertical texture evident in the IBED TiN is from thinning the sample during the FIB process. (Bottom) EDS of 1 of 3 Ti—Nb rich inclusions in the PVD TiNbN coating.

FIG. 7: SAED analysis of the three coating types in area closest to the Ti coupon/TiN coating interface, as well as in the bulk of the TiN coating thickness. The PVD coatings have similar d-spacing to IBED TiN near the interface, but in the bulk of the coating the ˜0.215 nm spacing is absent in the PVD coatings. PVD TiNbN and IBED TiN showed distinct changes in the first d-spacing between the interface and the bulk of the coating. The diffuse diffraction pattern of the IBED TiN demonstrates how much finer the grain structure is compared to the PVD coatings.

FIG. 8 includes an image showing an EDX mapping of a cross section of the porous coating with TiN IBED coating applied.

FIGS. 9A-9B show images starting from a zoomed out status (FIG. 9A) that depict a porous region and, when zoomed in (FIG. 9B), a specific area with a measurement of the thickness of a TiN coating. FIG. 9C is the same area measured in FIG. 9B but provides an EDX image confirming the measured area includes TiN.

FIGS. 10A-10D provide various view of an embodiment of a total knee prosthesis which may include regions discussed herein with regard to other embodiments (e.g., FIGS. 1E, 2, 8).

### DETAILED DESCRIPTION

Reference will now be made to the drawings wherein like structures may be provided with like suffix reference designations. In order to show the structures of various embodiments more clearly, the drawings included herein are diagrammatic representations of structures. Thus, the actual appearance of the fabricated structures, for example in a photo, may appear different while still incorporating the claimed structures of the illustrated embodiments (e.g., walls may not be exactly orthogonal to one another in actual fabricated devices). Moreover, the drawings may only show the structures useful to understand the illustrated embodiments. Additional structures known in the art may not have been included to maintain the clarity of the drawings. For example, not every layer of a device is necessarily shown. “An embodiment”, “various embodiments” and the like indicate embodiment(s) so described may include particular features, structures, or characteristics, but not every embodiment necessarily includes the particular features, structures, or characteristics. Some embodiments may have some, all, or none of the features described for other embodiments. “First”, “second”, “third” and the like describe a common object and indicate different instances of like objects are being referred to. Such adjectives do not imply objects so described must be in a given sequence, either temporally, spatially, in ranking, or in any other manner. A “first” element does not necessarily mean there is a “second” element. “Connected” may indicate elements are in direct physical or electrical contact with each other and “coupled” may indicate elements co-operate or interact with each other, but they may or may not be in direct physical or electrical contact. Phrases such as “comprising at least one of A or B” include situations with A, B, or A and B.

Unicompartmental knee femur fixation can fail by, for example, pistoning or an anterior rotation away from the distal cut surface of the knee. There is a trade-off between the correct alignment with the resulting cut surface boney coverage and the implant potentially interfering with patella tracking.

A traditional material for unicompartmental knee femurs is a CoCrMo alloy, however Cobalt or Chromium ions can leach out of CoCrMo implants and are known to cause physiological issues.

Orthopedic implants made from titanium alloy and which have articular surfaces have been tried in the past in both knee and hip arthroplasty. However, the surface hardness of these alloys was insufficient and their use resulted in wear debris related failures. After these failures, coating methods were developed in an effort to harden the articular surface. However, such coating methods do not produce a surface that is robust enough to perform equivalently to a CoCr implant, and as a result have not seen widespread use outside of addressing the needs of patients with a metal sensitivity or metal allergy.

Alternative non-cobalt chrome materials are available for orthopedic applications such as oxidized zirconium (OxZr) alloy or polyetheretherketone (PEEK). OxZr used in an articular implant is a surface oxidation layer of zirconium which does have excellent wear characteristics and can be formed into a number of implant types. However, it does not have the ability to support a porous ingrowth surface formed of the native OxZr nor applied porous titanium. PEEK has been formed into a number of orthopedic devices and can support a porous coating, however it is suboptimal as an articular surface.

In addition to the material-related issues above, year over year the number of patients needing a joint replacement has been increasing. In order to make this procedure more accessible and most cost effective, a universal component is desirable. However, achieving equivalent clinical function without left and right components creates additional constraints and challenges. Fixation, articulation, and boney coverage must all be considered and accommodated.

Embodiments present a novel approach to achieving an orthopedic implant with universal articulation, cementless fixation, and improved biocompatibility and wear characteristics. This approach can be applied to a unicompartmental knee femur as well as any other joint arthroplasty implant which has both a universal articular surface as well as a porous ingrowth surface for biological fixation.

An embodiment addresses an orthopedic knee prosthesis for a partial knee replacement with no left or right configurations required. Embodiments address porous coated implants for uncemented use. Embodiments address orthopedic total knee prostheses, orthopedic acetabular articular surfaces, glenoidal or reverse total shoulder implants, and/or articular spinal disk replacement devices.

An embodiment includes a universal unicompartmental knee femur. The embodiment includes cement pockets that are undercut in a direction which will resist pistoning and rotation away from the distal surface. These pockets allow for both cemented and cementless, porous coated, embodiments. An embodiment includes a pair of anterior pegs which resist anterior rotation. An embodiment has an articular surface that allows consistent rollback on a variety of size matching, ±100 of internal or external rotation without impinging on the patella tracking.

An embodiment utilizes a titanium alloy and ion bombarded surface treatment to allow the implant to be manufactured with both machining and additive methods and to be manufactured without using CoCrMo alloys.

In an embodiment, the bone contacting surface additionally has an applied porous coating to achieve biological fixation.

An embodiment can be formed into any orthopedic joint replacement implant geometry which has both an articular surface as well as a porous ingrowth surface. The porous surface would optimally be an applied coating, however the implant geometry and coating geometry could be achieved by, for example, metal additive manufacturing methods as well. The ion bombarded surface could contain metal or metalloid materials which improve either articular performance or produce an anti-microbial or anti-colonial effect.

Various examples are now addressed.

Example 1. A knee prosthesis, comprising: a unicondylar femoral implant (100) comprising a substrate (109), the substrate including a titanium alloy but not including titanium nitride; a first region (101) on/of the substrate, the first region including the titanium alloy and nitrogen (e.g., embedded nitrogen from IBED); a second region (102) on the first region, the second region including titanium nitride but not including the titanium alloy; a porous third region (103) on the substrate, the third region including grains of titanium but not including the titanium alloy; a fourth region (104) on the third region, the fourth region including titanium nitride but not including the titanium alloy; an articular surface (114) including the second region; a non-articular surface (113) including the third, and fourth regions; wherein: (a) the titanium alloy substrate is between the first and third regions, and (b) an axis (115) intersects the first region, the second region, the substrate, the third region, and the fourth region.

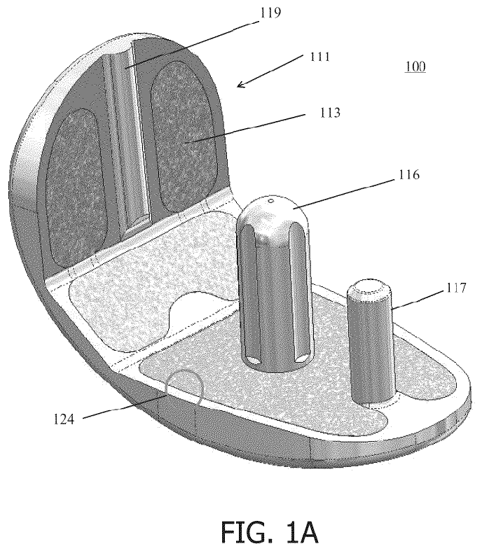

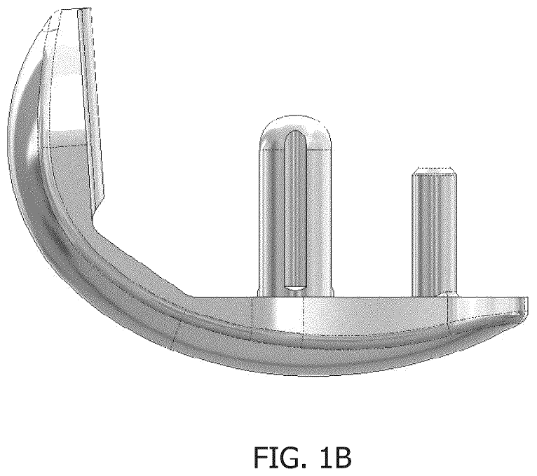

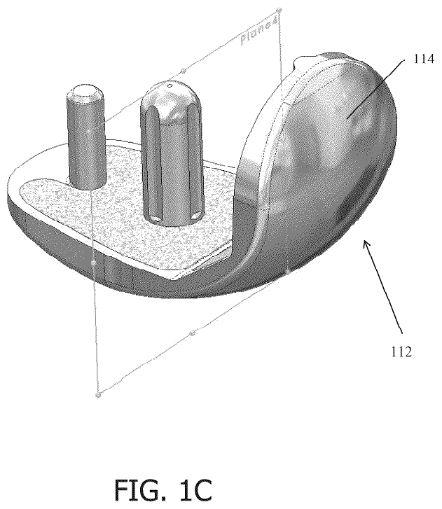

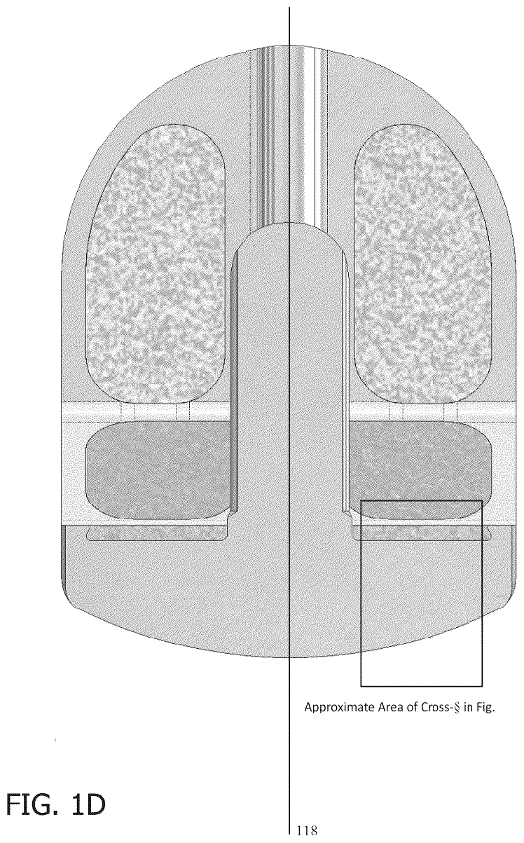

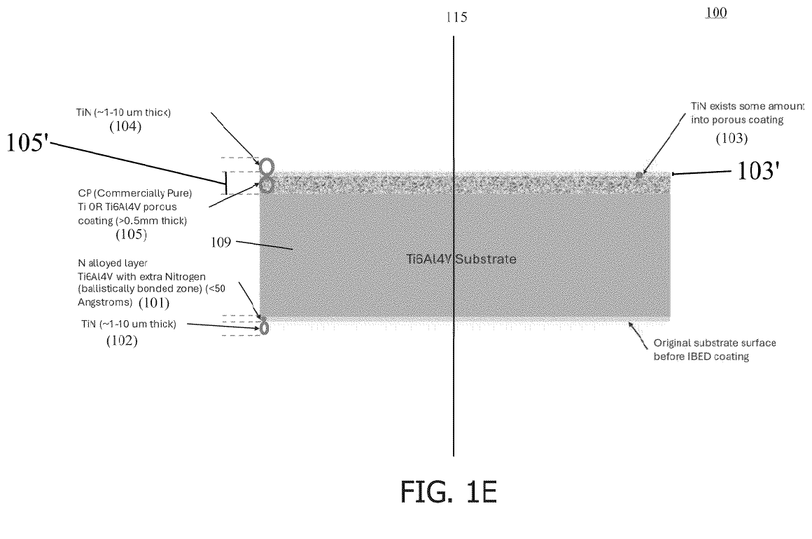

For instance see FIGS. 1A, 1B, 1C, 1D, and 1E. FIG. 1E is taken from the area so indicated on FIG. 1D.

Region 109 may include various materials such as, for example, Ti and Al and V (e.g., Ti6Al4v and/or Ti6Al4V ELI). Region 109 may include an alloy but in other embodiments may include any number of metals not necessarily including Ti.

Region 101 may “include N”. As used herein, this phase (and similar phrases providing a region “includes” an element) accounts for the presence of elemental N and/or N included in a composition such as titanium nitride (TixN1−x). Thus, region 101 may include N, TiN (without limit to any stoichiometric ratio between elements in a composition such as Ti and N) or other combinations based on what the substrate includes. In an embodiment, the N, in whatever form, is embedded into a region of the substrate via beam enhanced deposition (IBED). IBED is addressed in, for example, U.S. Pat. No. 9,523,144. Region 101 including N helps bond/adhere coating 102 to substrate 109, which is important because region 102 creates a surface with good wear and damage resistance properties.

Stating “first region (101) ON the substrate” includes the first region being within the substrate as, for example, an ion embedded region of the substrate that is adjacent other portions of the substrate to which ion embedding did not reach. Stating “(a) the titanium alloy substrate is BETWEEN the first and third regions” includes the portion of substrate that does not include embedded ions from, for example, IBED.

Region 102 may include a TiN surface having biocompatibility advantages. With the hardening provided in regions 101, 102, the TiN surface of region 102 provides increased wear resistance similar to CoCr but provides better biocompatibility than CoCr. While TiN is used in this embodiment, in other embodiments other elements or compositions may be used. For example, region 102 may include Ti and N or element A (not necessarily Ti or N) and element B (not necessarily Ti or N).

Region 103 has porosity that provides better ingrowth than materials like CoCr and is more biocompatible that CoCr. For example, bone has difficulty growing all the way to a surface with Co but will grow more aggressively to a surface with Ti due to titanium's osteogenesis properties. The IBED process may be applied to the side of the substrate that is eventually covered by the fourth region. Further, application of region 104 (e.g., TiN) to the porous structure of region 103 is very helpful for manufacturing because backside surface 111 does not need to be masked when applying region 102 to region 101, making fixturing much simpler and less custom (thereby decreasing manufacturing costs). Region 103 is the porous portion that also includes the TiN coating (region 104). The remainder of the porous portion (region 105) lacks the TiN coating because, for example, IBED is line of sight deposition and therefore TiN may not reach the porous portions (region 105) closest to substrate 109. See the circles in FIG. 1E showing how both 103 and 105 are porous (e.g., Ti grains) but 103 also includes TiN coating (104).

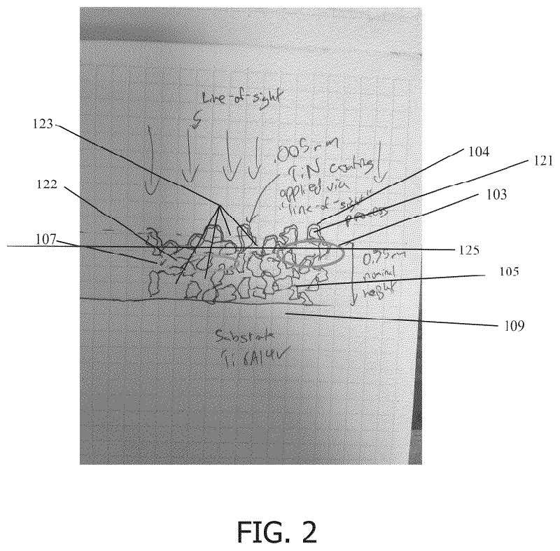

Region 104 has a surface finish of, for example, a TiN coating on the porous structure of region 103. Region 104 dips into region 103 (see circles in FIG. 1E for regions 103 and 104). FIG. 1E is abstract and my indicate region 104 to be a separate layer or region, however FIG. 2 is more realistic and shows region 104 to be a TiN coating on porous structures of region 103. Using a process such as IBED to apply the TiN coating (instead of, for example, phase vapor deposition (PVD)) is advantageous because the IBED process reproduces the underlying surface texture very faithfully, which helps retain the properties of region 103 for roughness, initial fixation, and the like.

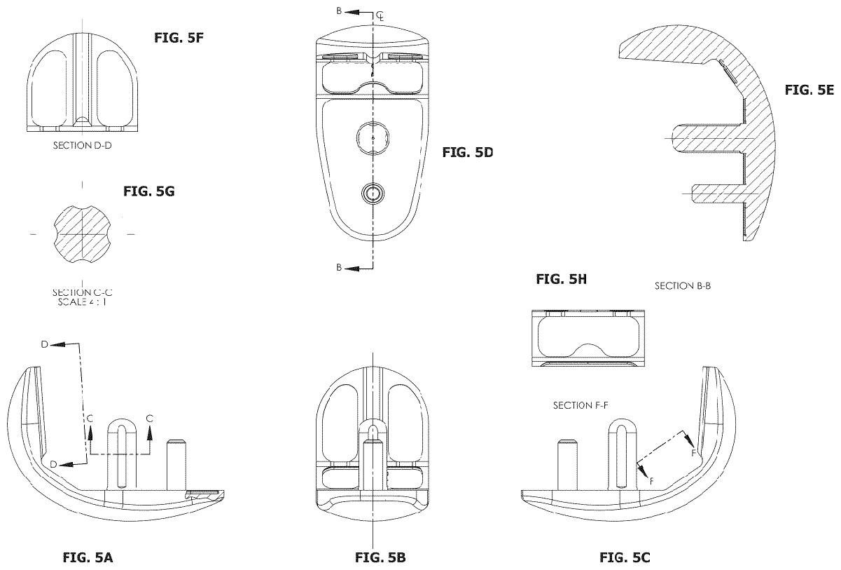

See also, for example, FIGS. 5A, 5B, 5C, 5D, 5E, 5F, 5G, 5H for another example of a unicondylar femoral implant including any of the same elements (or analogous elements) as described in Example 1.

Alternative version of Example 1. A knee prosthesis, comprising: a unicondylar femoral implant (100) comprising a substrate (109), the substrate including a titanium alloy but not including titanium nitride; a first region (101) on/of the substrate, the first region including the titanium alloy and titanium nitride; a second region (102) on the first region, the second region including titanium nitride but not including the titanium alloy; a porous third region (103) on the substrate, the third region including grains of titanium but not including the titanium alloy; a fourth region (104) on the third region, the fourth region including titanium nitride but not including the titanium alloy; an articular surface including the second region; a non-articular surface including the third, and fourth regions; wherein: (a) the titanium alloy substrate is between the first and third regions, and (b) an axis intersects the first region, the second region, the substrate, the third region, and the fourth region.

Thus, some embodiments provide region 102 with N (which may include elemental N and/or TiN among other N based compositions) while other embodiments may specify TiN is included in the second region.

Alternative version of 1. A knee prosthesis, comprising: a unicondylar femoral implant comprising a substrate, the substrate including a titanium alloy but not including titanium nitride; a first region on/of the substrate, the first region including the titanium alloy and titanium nitride; a second region on the first region, the second region including titanium nitride but not including the titanium alloy; a porous third region on the substrate, the third region including titanium but not including the titanium alloy; a fourth region on the third region, the fourth region including titanium nitride but not including the titanium alloy; an articular surface including the second regions a non-articular surface including the third, and fourth regions; wherein: (a) the titanium alloy substrate is between the first and third regions, and (b) an axis intersects the first region, the second region, the substrate, the third region, and the fourth region.

Thus, in some embodiments the third region does not necessarily include Ti grains but may include Ti formed via, for example, additive manufacturing and the like wherein the Ti is not necessarily disposed as grains.

Another version of 1. A knee prosthesis, comprising: a unicondylar femoral implant (100) comprising a substrate (109), the substrate including an alloy and not including titanium nitride; a first region (101) on/of the substrate, the first region including the alloy and nitrogen; a second region (102) on the first region, the second region including titanium and nitrogen but not including the alloy; a porous third region (103) on the substrate, the third region including titanium but not including the alloy; a fourth region (104) on the third region, the fourth region including titanium and nitrogen but not including the alloy; an articular surface (114) including the second region; a non-articular surface (113) including the third, and fourth regions; wherein: (a) the alloy is between the first and third regions, and (b) an axis (115) intersects the first region, the second region, the substrate, the third region, and the fourth region.

Another version of Example 1. A knee prosthesis, comprising: an implant (100) comprising a substrate (109), the substrate including an alloy; a first region (101) on/of the substrate, the first region including the alloy and nitrogen; a second region (102) on the first region, the second region including titanium and nitrogen but not including the alloy; a porous third region (103) on the substrate, the third region including titanium but not including the alloy; a fourth region (104) on the third region, the fourth region including titanium and nitrogen but not including the alloy; an articular surface (114) including the second region; a non-articular surface (113) including the third, and fourth regions; wherein: (a) the alloy is between the first and third regions, and (b) an axis (115) intersects the first region, the second region, the substrate, the third region, and the fourth region.

Alternative version of Example 1. A knee prosthesis, comprising: a unicondylar femoral implant (100) comprising a substrate (109), the substrate including a titanium alloy but not including titanium nitride; a first region (101) on/of the substrate; a second region (102) on the first region, the second region including titanium nitride but not including the titanium alloy; a porous third region (103) on the substrate, the third region including grains of titanium but not including the titanium alloy; a fourth region (104) on the third region, the fourth region including titanium nitride but not including the titanium alloy; an articular surface (114) including the second region; a non-articular surface (113) including the third, and fourth regions; wherein: (a) the titanium alloy substrate is between the first and third regions, and (b) an axis (115) intersects the first region, the second region, the substrate, the third region, and the fourth region.

In some embodiments, region 101 may be 50 Angstroms or less in thickness. In other embodiments, region 101 may be 10-20 Angstroms in thickness. The thinness of the region may lead to difficulty in imaging the region to distinguish the region from other regions.

Circles are included in FIG. 1E to demonstrate the relationship between regions 101, 102, 103, 105, 105, 109. The following showing ranges of thickness/length (measured parallel to axis 115) for embodiments:

Region102: IBED TiN Articular Surface Coating: 1-10 microns

Region 101: N-Alloyed region: 0-50 Angstroms. Some embodiments may be, for example, between 10-20 Angstroms.

Thickness 105′: Porous coating/region (inclusive of TiN coated region 103 and non-TiN coated Ti region 105): 0.5-1.5 mm. Embodiments with a sintered coating/region may be: 0.5-1 mm. However, in other embodiments 105′ may be much thicker (e.g., with additive manufacturing techniques) an may, for example, extend to 3 mm or so in thickness (all of which may vary based on the geometric and strength requirements of the implants and the coatings).

Thickness 103′ (which is the thickness of region 103): This thickness may vary in embodiments depending on the structure of the coating/porous region. Some structures, especially additively manufactured surfaces, may allow TiN 104 to be applied all the way down to the substrate in some locations. This could occur if there were a pore in the coating open at the top which runs straight through to the substrate 109.

If a pore were to extend through the entire porous coating all the way to the substrate and TiN is formed within the pore all the way to the substrate, region 105 may be said to not exist in that particular part the device. However, a short distance away from that pore other pores may not extend through the entire porous coating all the way to the substrate and in that area with these other shorter pores, region 105 may be said to exist. In other words, for example, a small minority of areas without region 105 do not necessarily mean the entire device is without region 105.

104: TiN coating applied on top of the outermost grains: 0.5-10 microns. Region 104 in various embodiments may have sub micron thickness (e.g., as region 105 may be, in some embodiments of a manufacturing method, receiving the “overspray” of the TiN coating that was aimed at the articular surface). Other embodiments could include intentionally thickening this coating.

Alternative version of Example 1. A knee prosthesis, comprising: a unicondylar femoral implant (100) comprising a substrate (109), the substrate including a titanium alloy but not including titanium nitride; a first region (102) on the substrate, the first region including titanium nitride but not including the titanium alloy; a porous second region (103) on the substrate, the second region including grains of titanium but not including the titanium alloy; a third region (104) on the second region, the third region including titanium nitride but not including the titanium alloy; an articular surface (114) including the first region; a non-articular surface (113) including the second, and third regions; wherein: (a) the titanium alloy substrate is between the first and second regions, and (b) an axis (115) intersects the first region, the second region, the substrate, the second region, and the third region.

In some embodiments, region 101 may be 50 Angstroms or less in thickness. In other embodiments, region 101 may be 10-20 Angstroms in thickness. The thinness of the region may lead to difficult in imaging the region to distinguish the region from other regions.

Example 1.1 The knee prosthesis of Example 1, wherein the porous third region is included in a pocket and the outermost surface of the porous third region is coplanar with an outer wall that defines the pocket.

For example, see area 124 of FIG. 1A.

2. The knee prosthesis according to any of Examples 1-1.1 comprising first and second posts (116, 117) that interface the non-articular surface.

Such posts may be used to fasten the implant to bone, such as the femur. However, other embodiments may include no such posts or a sing such post.

Example. The knee prosthesis of Example 2, wherein a plane (118, FIG. 1D) bisects the unicondylar femoral implant and intersects the first and second posts.

Example 3. Alternative version: The knee prosthesis of Example 2, wherein a plane (118) intersects the first and second posts.

Example 4. The knee prosthesis of Example 3, wherein the plane intersects the substrate and the first, second, third, and fourth regions.

Example 5. The knee prosthesis according to any of Examples 2-3, comprising a protuberance (119, FIG. 1A) that interfaces the non-articular surface.

A protuberance, such as linear ridge 119, may interface a trough cut/formed into the posterior condyle of the femur bone, providing additional rotational stability. However, other such embodiments may lack the protuberance.

Example 6. The knee prosthesis of Example 5, wherein the protuberance is linear and the plane intersects the protuberance.

Example 7. The knee prosthesis according to any of Examples 3-6, wherein the articular surface is symmetric about the plane.

This symmetry allows the device to be universal (symmetric and not left knee or right knee specific). However, other embodiments are arranged as non-symmetric implants.

Example 8. The knee prosthesis according to any of Examples 1-7, wherein the first region and the third region both directly contact the substrate. Again, region 101 may be an ion embedded portion of the substrate.

For example, an embodiment of an IBED-based method includes placing the substrate (substrate 109) (possibly with porous region 103 already present from having previously sintered, additive manufacturing, etc. a material onto the substrate) in a vacuum chamber and then performing ion beam sputtering on the exposed surfaces, which cleans and texturizes the surfaces. Coating also occurs (possibly in parallel with the texturing/cleaning) to embed, for example, N to form region 101 (e.g., TiAlV from substrate and N, possibly as elemental N or TiN), 102 (e.g., TiN) as well as 104 (e.g., TiN). The parts being coated may be rotated within the chamber along one or multiple axes of rotation in order to evenly coat all surfaces. The direct contact between, e.g., region 101 and the substrate is a result of this example process.

Example 9. The knee prosthesis of Example 8, wherein the second region directly contacts the first region, and the fourth region directly contacts the third region.

Example 10. The knee prosthesis according to any of Examples 1-9, wherein the articular surface is universal and not particularly configured for either of right or left knees.

Example 11. The knee prosthesis according to Examples 1-10, wherein the grains of titanium are sintered to the substrate.

Sintering the grains is one method of adhering the grains to the substrate. However, other embodiments are not so limited. For example, Ti (in grain form or any other form) may be formed via additive manufacturing.

Sintered material may show grain growth in the substrate material from heat treatments required to get the Ti of region 103 and substrate 109 to bond together.

As addressed in “The effect of post-sintering heat treatments on the fatigue properties of porous coated Ti-6Al-4V Alloy”, Journal of Biomedical Materials Research, Volume 22, Issue 4 (April 1988) Pages 287-302, porous coated Ti-6Al-4V alloy implant systems provide a biocompatible interface between implant and bone, resulting in firm fixation and potential long-term retention via bony ingrowth. In order to achieve an acceptable porous coating structure, the sintering protocol for Ti-6Al-4V alloy systems often requires that the material be heat treated above the beta transus. This transforms the as-received equiaxed microstructure, recommended for surgical implants, to a lamellar alpha-beta distribution, which has been shown to have poor fatigue properties of the most common structures attainable in Ti-6Al-4V alloy. However, post-sintering heat treatments may be used to improve these properties by producing microstructures more resistant to crack initiation and propagation.

An embodiment has sintered material that is subjected to a BUS “Broken up Structure” treatment, similar to the following. As addressed in “Sintering Temperature Effects On the Mechanical Properties of Porous-Coated Ti-6al-4v Eli Alloy”, Jeff Archbold, Thesis at University of Toronto, 1999, slow cooling (1 degree C./min) from temperatures well above 992 degrees C. results in a coarse lamellar structure termed beta-annealed (BA). It is also possible to form other variants of this microstructure designated as basket-weave structures. One way to create a basket-weave microstructure involves heating the titanium alloy above the alpha beta allotropic transformation temperature and then quenching. The titanium alloy is then held in the alpha range, usually at BOO′C for 24 hours. This treatment creates a broken-up-structure (BUS), a base microstructure of beta-phase grains with interspersed alpha-phase. The BUS microstructure exhibits improved fatigue characteristics versus notched MA microstructures and both un-notched and notched BA microstructures.

Example 12. The knee prosthesis according to any of Examples 1-11, wherein the knee prosthesis includes no chromium and no cobalt.

Alternative version of Example 12. The knee prosthesis according to any of Examples 1-11, wherein the knee prosthesis includes less than 2% composition of chromium and less than 2% composition of cobalt.

Thus, the prosthesis may have no chromium or cobalt or only trace amounts of the same. This purity may be key considering there may be biocompatibility issues with Cobalt.

Example 12.1. The knee prosthesis according to any of Examples 1-11, wherein the knee prosthesis includes no nickel.

Alternative version of Example 12.1. The knee prosthesis according to any of Examples 1-11, wherein the knee prosthesis includes less than 2% composition of nickel.

By forming devices that are hardened to lessen wear, the devices can avoid metals such as chromium, cobalt, nickel, and the like avoid metal allergies and biocompatibility issues.

Example 13. The knee prosthesis according to any of Examples 1-12, wherein: the non-articular surface includes a lip with a recessed surface that forms a void; an axis extends through the lip, the void, and the substrate; the void is between the lip and substrate.

As a result, if bone cement (or analogous material) is used to couple the device to bone, the bone cement can flow or be otherwise introduced into the void and thereby better couple the device to bone. However, in other embodiments the void is filled with porous coating material such as, for example, sintered Ti.

Example 14. The knee prosthesis according to any of Examples 1-13, wherein at least one of the grains of titanium has a length of between 180 microns and 500 microns.

Such a grain size may be critical in some embodiments and may lead to an appropriate pore size for bone ingrowth.

Example 15. The knee prosthesis according to any of Examples 1-14, wherein the substrate does not include nitrogen.

Alternative version of Example 15. The knee prosthesis according to any of Examples 1-14, wherein the substrate includes less than 2% nitrogen.

In other embodiments the substrate includes less than 1.5%, 1%, 0.05% (e.g., Titanium F136 or F1472).

Example 16. The knee prosthesis according to any of Examples 1-15, wherein the first region is compressively stressed.

For example, ion embedding may embed N into the first region to create compressive stress.

Example 17. The knee prosthesis of Example 16, wherein the third region is compressively stressed.

Example 18. The knee prosthesis according to any Examples 1-17, wherein the porous surface includes a pore (123) between two or more grains of the titanium.

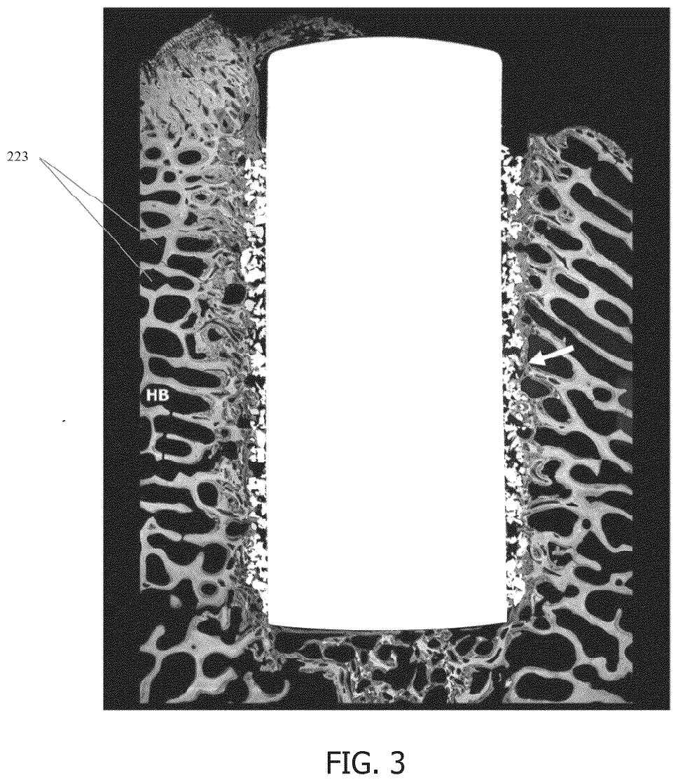

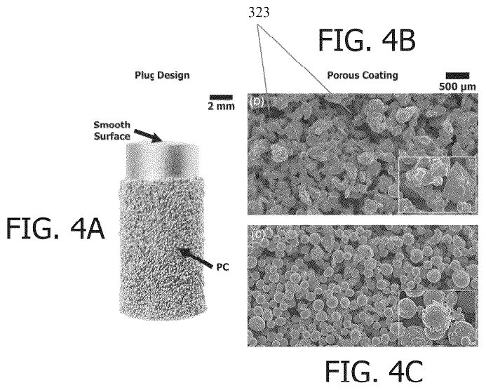

See also, for example, pores 223 in FIG. 3 (which is a porous Ti coating on a plug), or pores 323 in FIG. 4B. FIG. 3 is a cross-sectional image of a porous Ti coating applied to a small 14″ pin. FIG. 4B is an SEM image of a porous coating (without TiN), as well as an image of the pin of FIG. 3. FIG. 4B shows Ti grains of, for example, 100 to 500 microns.

Example 19. The knee prosthesis of Example 18, wherein the mean pore size is between 135 and 400 microns in diameter.

Such pore sizes may be critical in that they are optimal for bony ingrowth

Example 20. The knee prosthesis of Example 18, wherein the pore extends through the third and fourth regions and contacts the substrate.

As used herein, the “third region” (or any other “region” addressed herein) may be in the form of a general area or a discrete layer. A layer may be uniform or may be comprised of multiple layers (e.g., a laminate construction). For example, FIG. 1E is abstract and shows regions that appear as layers of consistent thickness and the like. However, FIG. 2 shows how region 104 may be a layer or region that resembles a coating of varying thickness and which is non-planar. The layer may be thicker where line-of-sight is direct but may be thinner within voids where deposition/embedding is more difficult to reach.

Example 20.1 The knee prosthesis of Example 18, wherein the porous surface includes interconnected pores.

This may only be the case for a small subset of the pores. An embodiment may include a porous surface that has interconnected pores, such that the bone can grow in and “behind” metal porous structures. This provides better hold for the bone/implant.

Example 21. The knee prosthesis of Example 20, wherein the pore is hollow.

For example, the pore is not filled with any material thereby providing space for bone ingrowth.

Example 21.1 The knee according to any of Examples 18-21, wherein an additional axis (125) intersects the third region, the fourth region, at least one of the pores.

Example 22. The knee prosthesis of Example 18, wherein the porous surface is at least 0.50 mm thick.

Such a thickness may be critical to give sufficient adherence to the bone once the bone is grown into the pores. Embodiments include a thickness, measured parallel to axis 115, between 0.50-0.75 mm or between 0.75-1.5 mm. Forming the porous region too thickly weakens the region.

Example 23. The knee prosthesis according to any of Examples 1-22 comprising a porous fifth region (105) on the substrate, wherein: the fifth region includes the grains of titanium; the fifth region is between the substrate and the third region.

Alternative version of Example 23. The knee prosthesis according to any of Examples 1-22 comprising a porous fifth region (105) on the substrate, the fifth region including the grains of titanium.

See, for example, FIG. 2. At the bottom of the TiN/Ti Porous coating interface (region 104/103 interface) on each bead there may be a N enriched/alloyed area (107). The thickness of the TiN (region 104) is exaggerated in FIG. 2. Thus, FIG. 2 shows a TiN coating 104 that coats a Ti grain 121. The coating itself in region 104 is TiN. Region 103 includes Ti grains. Region 107 may include Ti grains and elemental N (122). Area 105 may include Ti grains but no nitrogen. Area 109 may include the substrate, such as a Ti alloy. Pores 123 are located within the porous coating.

Region 103 includes a porous structure. Region 104 is added to, for example, element 121 of region 103 while retaining the subsurface structure of element 121. This is due to, for example, the nanocrystalline nature of the TiN coating of region 104. As a result, the roughness of the porous structure (such as that found in areas 103, 105) is retained even as it is covered with, for example, a 5 micron layer of TiN.

In an embodiment, the TiN of region 104 covers the top of the porous structure, but does not fill up the pores such as pores 123. Region 104 will penetrate some distance into the porous coating (e.g., on the top faces of the coating which are “exposed” to the outside).

Example 23.1 The knee prosthesis according to the alternative version of Example 23, wherein the fifth region includes less than 2% composition of nitrogen.

Example 24. The knee prosthesis according to any of Examples 1-23.2 wherein the grains of titanium are arranged asymmetrically with respect to each other.

Another version of Example 24. The knee prosthesis according to any of Examples 1-23.1 wherein the grains of titanium are asymmetrically shaped.

For example, the Ti grains are non-spherical. For example, see FIGS. 4A, 4B, 4C. FIG. 4A shows a plug having a Ti porous coating. FIG. 4B is a closeup of Ti grains forming a Ti coating. FIG. 4C shows, in contrast to asymmetric Ti grains, spherical CoCr porous coating.

Spherical beads may be problematic considering they may be so smooth that bone does not readily adhere to them and grow directly on them. Also, spherical beads may not create a rough external surface, so typically have lower coefficient of friction against bone. This can lead to more motion in the early phases of implantation when the bone is trying to grow in, and potentially impacting ingrowth

Example 25. The knee prosthesis according to any of Examples 1-24 comprising a tibial implant.

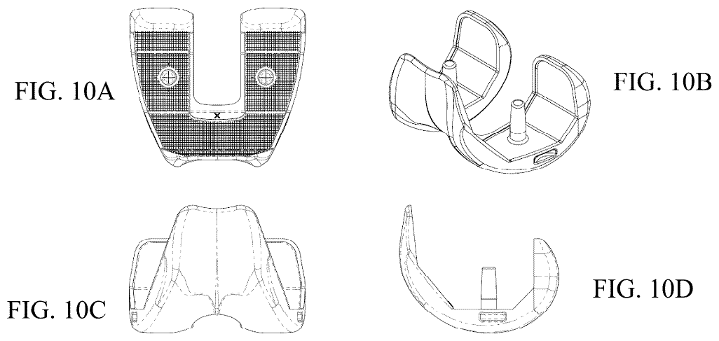

While embodiments included herein may focus on one particular type of implant, other embodiments are not so limited and may include, for example, a total knee prosthesis (see, e.g., FIG. 10A). In FIG. 10A, the hashed portion may include a porous area like regions 105, 103. In such a case, the femoral component may have a different geometry from that shown in FIG. 1A and may cover multiple condyles (e.g., anterior, distal, posterior) and the whole anterior flange with metal, as opposed to just the single distal and posterior condyle that is covered by the implant of FIG. 1A. However, other elements such as the regions addressed in Example 1 may be similar, regardless whether it is a total knee implant or a unicondylar implant.

Embodiments addressed herein include a porous region but in other embodiments the porous region may be omitted. For instance, region 104 may be formed directly on the substrate. Bone cement may be used in such a situation to couple the implant to bone. Region 103 may exist in the outermost edge of the substrate such that region 103 includes, for example, nitrogen and an alloy of the substrate.

Example 26. The knee prosthesis according to any of Examples 1-25 wherein the unicondylar femoral implant is configured for cementless fixation to a patient's femur.

Example 27. The knee prothesis according to any of Examples 1-26 wherein the substrate is non-porous.

Example 28. The knee prosthesis according to any of Examples 1-27 wherein the second region is less than 15 microns thick.

For example, this dimension may be critical because if region 102 is too thick the layer or region may become brittle and prone to fracture. Other embodiments are less than 12, 10, 8, or 6 microns.

Example 28.1 The knee prosthesis according to any of Examples 1-28 wherein the third region is between 50-100 Angstroms thick as measured parallel to the axis (115).

As used herein, thickness may also be considered “length” and is taken parallel to axis 115. Region 103 (of FIG. 1E) includes thickness 103′.

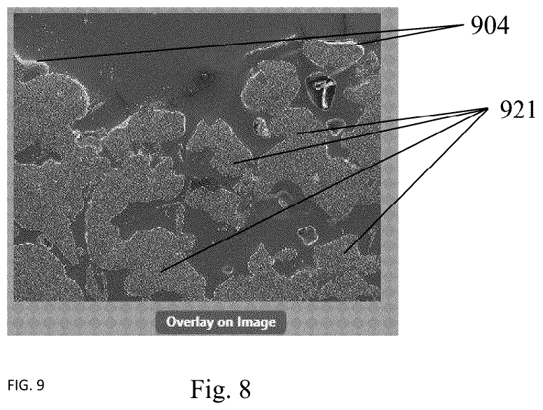

FIG. 8 shows an EDX mapping of a cross section of the porous coating with TiN IBED coating applied (portion 104 in FIGS. 1E/2). Titanium grains 921 exist and TiN coating 904 is mainly applied to the tops of the grains. This shows how variable/rough the thickness of the porous coating is.

In an embodiment the porous region as a whole is ˜0.5-1.5 mm thick (see regions 103 plus 105 of FIG. 1E and thickness 105′). The grains of titanium that make up this porous region are coated in ˜0.5-10 microns of TiN (e.g., region 104 in FIG. 2). This layer of TiN (e.g., 904 in FIG. 8) is applied to the grains (e.g., 921) via a line of sight process (IBED), so it is thicker on the top of the grains and thinner, or non-existent, on the backside of the grains. Also, deeper into the porous coating (e.g., area 105 near the substrate) where it is more shielded, the TiN is not present. Thickness 105′ may be 0.7 mm to 0.9 mm in other embodiments. In an embodiment, tops of the porous region that are exposed to IBED may have ˜0.5-10 micron of TiN (104) on them. Underneath this TiN, embodiments may include a very thin layer of N-alloyed Ti (not shown), helping to adhere the TiN 914 to the Ti grain 921.

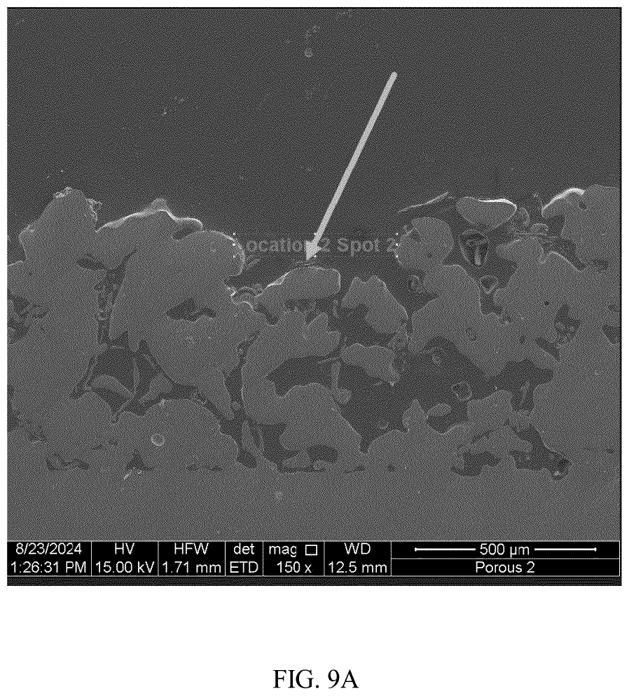

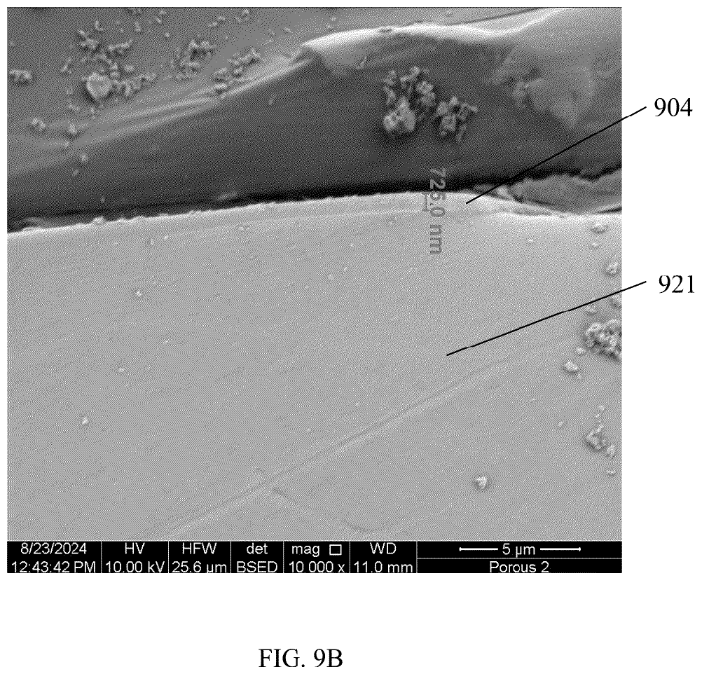

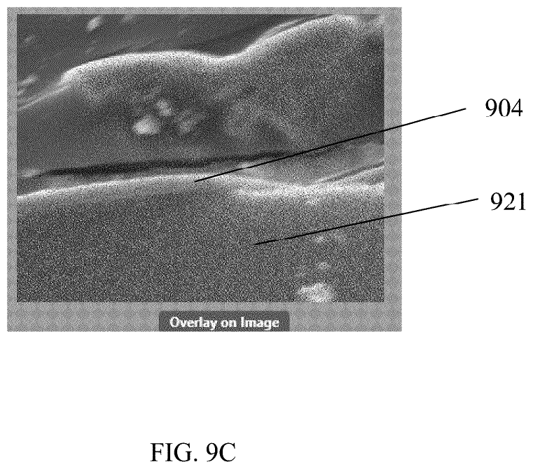

FIGS. 9A and 9B show increasing magnifications of region 103. FIGS. 9B and 9C show a Ti grain 921 with a TiN coating 904. FIG. 9B shows the thickness of TiN 904. FIG. 9C is the same area as FIG. 9B but uses EDX to confirm area 904 is TiN.

Example 29. The knee prosthesis according to any of Examples 1-28.1, wherein the second region consists essentially of titanium nitride.

For example, in the manufacturing process the region is formed in a vacuum and, consequently, only Ti and N are applied.

Example 30. The knee prosthesis according to any of Examples 1-29, wherein: the second region includes first and second subregions; the first subregion of the second region is between the first region and the second subregion of the second region; the second subregion of the second region has more nitrogen and then the first subregion of the second region.

In other words, as the IBED process is performed the concentration of titanium nitride decreases as embedding ions go further into the substrate.

Example 31. The knee prosthesis of Example 30 comprising a sixth region, wherein: the second region is between the first and sixth regions; the sixth region includes first and second subregions; the first subregion of the sixth region is between the second region and the second subregion of the sixth region; the second subregion of the sixth region has more nitrogen and then the first subregion of the sixth region.

For example, the IBED process may occur repeatedly thereby depositing the sixth region onto and within the second region. As a result, there is a two layer ceramic coating on at least one side of the substrate. This is not shown in the Figures. The sixth and second regions may include different materials. For example, one of the layers may include silver or similar material having antimicrobial properties. One of the IBED formed areas may include, for example, SiN while another IBED region includes TiN or some other material.

Applicant engaged in a study as follows.

Hard ceramic coatings such as Physical Vapor Deposition (PVD) TiN, PVD TiNbN, and Ion Beam Enhanced Deposition (IBED) TiN may be utilized in orthopedics to improve the wear and durability of Titanium articular surfaces, and to reduce the leaching of metal ions from Cobalt Chrome implants. Applicant conducted a study using TEM to investigate crystallographic characteristics of the different coatings.

### Methods:

Polished Titanium coupons (1.312″ diameter, Ti6Al4V) were coated with a nominal 5-micron-thick layer of either PVD TiN, PVD TiNbN, or IBED TiN. Cross-sectional specimens were prepared using Focused Ion Beam (FIB) milling to produce lamellae approximately 7×10 m in size and 100-300 nm thick. High-resolution imaging was performed using a JEOL JEM-2800 S/TEM operating at 200 kV Both TEM and STEM modes were utilized, including bright field, dark field, and secondary electron imaging. Crystallography was analyzed using selected area electron diffraction (SAED). ImageJ software was used to manually estimate the grain size.

### Results:

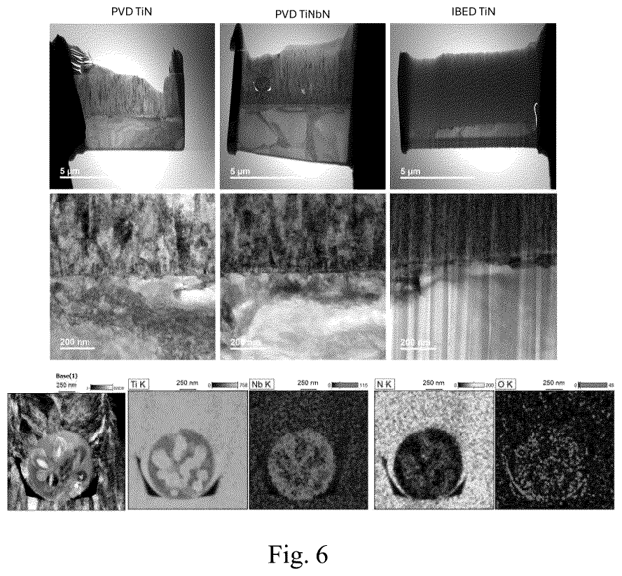

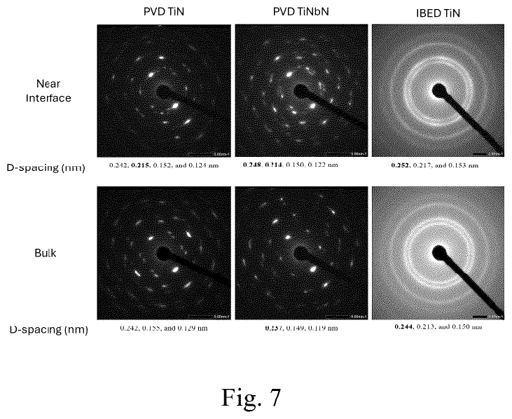

IBED TiN coatings exhibited a uniform nanocrystalline grain structure with grain size on the order of 10 nm, consistent throughout the coating thickness. In contrast, both PVD TiN and PVD TiNbN coatings demonstrated coarse, overlapping columnar grains oriented perpendicular to the interface. For both PVD TiN and PVD TiNbN, the grains were on the order of 500-3000 nm long by 50-500 nm wide in the bulk coating, and 10-50 nm wide by 50-500 nm long at the coating-substrate interface, with the smallest grains adjacent to the interface (FIG. 6). SAED showed similar d-spacing between all coatings near the interface, but in the bulk of the thickness, the 0.215 nm spacing was absent from the PVD coatings. The first d-spacing of both PVD TiNbN and IBED TiN are distinctly different between the interface and bulk coating (FIG. 7). The diffuse diffraction pattern of the IBED TiN clearly demonstrates how much finer the grain structure is compared to the PVD coatings. Notably, the PVD TiNbN sample contained significant defects in the coating: one void and two Niobium-rich inclusions surrounded by additional voids.

### CONCLUSIONS

The IBED TiN coating demonstrated a more refined and uniform structure with consistent crystallographic features, suggesting improved interfacial stability and mechanical performance. The PVD coatings have an additional crystal phase and much finer grain size near the substrate interface, demonstrating that the crystal structure of these coatings changes significantly throughout the thickness. The presence of defects in the PVD TiNbN coating has been previously shown and may present some concern for durability. These morphological differences are likely due to the coating methods. Possibly the line-of-sight formation with IBED is at least partially responsible for the morphological differences. PVD coatings are created by arc-sputtering of Titanium and/or Niobium targets into an Argon-Nitrogen plasma, followed by deposition onto the substrate surface, a process which creates droplets that precipitate directly onto the surface. In IBED, Titanium atoms are electron gun evaporated in vacuum, and the coating is formed directly on the substrate using an energetic nitrogen ion beam. The stark difference in grain structure and presence of defects in the PVD coatings compared to the IBED coatings can most likely be attributed to the difference in coating method. These results demonstrate the importance of coating characterization and suggest that the deposition method plays a critical role in determining coating integrity and morphology.

Example 32. The knee prosthesis of Example 1, wherein the titanium nitride of the fourth region includes a nanocrystalline grains between 0 and 20 nm in length as measured parallel to the axis.

These ranges may also apply to nanocrystalline grains of region 102.

However, in other embodiments the grains are between 0 and 30 nm, 0 and 40 nm, or 0 and 50 nm. Other embodiments are not constrained by the length of the nanocrystalline grains. This length may be critical in promoting better interfacial stability, coating integrity, and mechanical performance.

Example 23. The knee prosthesis of Example 22, wherein the nanocrystalline grains are non-overlapping with one another.

Example 24. The knee prosthesis of Example 22, wherein: the fourth region includes first and second halves; the first half of the fourth region is between the third region and the second half of the fourth region; the nanocrystalline grains of the first half of the fourth region have a first total number of crystal phases; the nanocrystalline grains of the second half of the fourth region have a second total number of crystal phases; the first total number of crystal phases is equal to the second total number of crystal phases.

See, for example, FIG. 7. The consistency in crystal phase, as opposed to PVD methods, may be critical in promoting better interfacial stability, coating integrity, and mechanical performance.

The foregoing description of the embodiments of the invention has been presented for the purposes of illustration and description. It is not intended to be exhaustive or to limit the invention to the precise forms disclosed. This description and the claims following include terms, such as left, right, top, bottom, over, under, upper, lower, first, second, etc. that are used for descriptive purposes only and are not to be construed as limiting. For example, terms designating relative vertical position refer to a situation where a side of a substrate is the “top” surface of that substrate; the substrate may actually be in any orientation so that a “top” side of a substrate may be lower than the “bottom” side in a standard terrestrial frame of reference and still fall within the meaning of the term “top.” The term “on” as used herein (including in the claims) does not indicate that a first layer “on” a second layer is directly on and in immediate contact with the second layer unless such is specifically stated; there may be a third layer or other structure between the first layer and the second layer on the first layer. The embodiments of a device or article described herein can be manufactured, used, or shipped in a number of positions and orientations. Persons skilled in the relevant art can appreciate that many modifications and variations are possible in light of the above teaching. Persons skilled in the art will recognize various equivalent combinations and substitutions for various components shown in the Figures. It is therefore intended that the scope of the invention be limited not by this detailed description, but rather by the claims appended hereto.

1. A knee prosthesis, comprising:
a femoral implant comprising a substrate, the substrate including a titanium alloy but not including titanium nitride;
a first region of the substrate, the first region including the titanium alloy and nitrogen;
a second region on the first region, the second region including titanium nitride but not including the titanium alloy;
a porous third region on the substrate, the third region including grains of titanium but not including the titanium alloy;
a fourth region on the third region, the fourth region including titanium nitride but not including the titanium alloy;
an articular surface including the second region;
a non-articular surface including the third and fourth regions;
wherein: (a) the titanium alloy of the substrate is between the first and third regions, and (b) an axis intersects the first region, the second region, the substrate, the third region, and the fourth region.

2. The knee prosthesis of claim 1, comprising first and second posts that interface the non-articular surface, wherein:
the third region is included in a pocket and an outermost surface of the third region is coplanar with an outer wall that defines the pocket.

3. The knee prosthesis of claim 2, wherein:
a plane bisects the femoral implant and intersects the first and second posts;
the plane intersects the substrate and the first, second, third, and fourth regions;
the femoral implant is a unicondylar femoral implant.

4. The knee prosthesis of claim 3, comprising a protuberance that interfaces the non-articular surface, wherein the protuberance is linear and the plane intersects the protuberance.

5. The knee prosthesis of claim 3, wherein the articular surface is symmetric about the plane.

6. The knee prosthesis of claim 1, wherein:
the first region and the third region both directly contact the substrate;
the second region directly contacts the first region, and the fourth region directly contacts the third region.

7. The knee prosthesis of claim 1, wherein the grains of titanium are sintered to the substrate.

8. The knee prosthesis of claim 1, wherein the knee prosthesis includes no chromium, no cobalt, and no nickel.

9. The knee prosthesis of claim 1, wherein:
the non-articular surface includes a lip and a recessed surface that collectively form a void;
an additional axis extends through the lip, the void, and the substrate;
the void is between the lip and the substrate.

10. The knee prosthesis of claim 1, wherein at least one of the grains of titanium has a length of between 180 microns and 500 microns.

11. The knee prosthesis of claim 1, wherein:
the first region is compressively stressed;
the third region is compressively stressed.

12. The knee prosthesis of claim 1, wherein:
the third region includes a pore between two or more grains of the titanium;
a mean pore size of pores of the third region is between 135 and 400 microns in diameter;
the pore extends through the third and fourth regions and contacts the substrate.

13. The knee prosthesis of claim 12, wherein the pore is hollow.

14. The knee prosthesis of claim 13, wherein:
an additional axis intersects the third region, the fourth region, at least one of the pores;
the third region is at least 0.50 mm thick.

15. The knee prosthesis of claim 1 comprising a porous fifth region on the substrate, wherein:
the fifth region includes the grains of titanium;
the fifth region is between the substrate and the third region;
the fifth region includes less than 2% composition of nitrogen.

16. The knee prosthesis of claim 1, wherein the grains of titanium are asymmetrically shaped.

17. The knee prosthesis of claim 1, wherein:
the femoral implant is configured for cementless fixation to a patient's femur;
the substrate is non-porous;
the second region is less than 15 microns thick.

18. The knee prosthesis of claim 1, wherein the second region consists essentially of titanium nitride.

19. The knee prosthesis of claim 1, wherein:
the second region includes first and second subregions;
the first subregion of the second region is between the first region and the second subregion of the second region;
the second subregion of the second region has more nitrogen than the first subregion of the second region.

20. The knee prosthesis of claim 19 comprising a sixth region, wherein:
the second region is between the first and sixth regions;
the sixth region includes first and second subregions;
the first subregion of the sixth region is between the second region and the second subregion of the sixth region;
the second subregion of the sixth region has more nitrogen and then the first subregion of the sixth region.

21. The knee prosthesis of claim 1, wherein the titanium nitride of the fourth region includes nanocrystalline grains between 0 and 20 nm in length as measured parallel to the axis.

22. The knee prosthesis of claim 21, wherein:
the fourth region includes first and second halves;
the first half of the fourth region is between the third region and the second half of the fourth region;
the nanocrystalline grains of the first half of the fourth region have a first total number of crystal phases;
the nanocrystalline grains of the second half of the fourth region have a second total number of crystal phases;
the first total number of crystal phases is equal to the second total number of crystal phases.

23. The knee prosthesis of claim 21, wherein the titanium nitride of the second region includes nanocrystalline grains between 0 and 20 nm in length as measured parallel to the axis.
